(19) United States
(12) Patent Application Publication
(10) Pub. No.: US 2023/0152301 A1
(43) Pub. Date: May 18, 2023

(54) MICRO-FLUIDIC DEVICE AND MODULE, MANUFACTURING METHOD THEREOF , AND METHOD FOR TESTING REACTIVITY OF CANCER CELLS TO ANTI-CANCER DRUG

(71) Applicant: NC CO., LTD., Cheongdo-gun (KR)

(72) Inventor: Chun Gwang BYUN, Seoul (KR)

(21) Appl. No.: 17/542,771

(22) Filed: Dec. 6, 2021

(30) Foreign Application Priority Data
Nov. 17, 2021 (KR) 10-2021-0158829

(51) Int. Cl. G01N 33/50; B01L 3/00

(52) U.S. Cl. CPC G01N 33/5011; B01L 3/502753; B01L 3/502707; B01L 2300/088; B01L 2200/0652; B01L 2200/12

(57) ABSTRACT

The present invention relates to a spiral microfluidic device and module for CTC separation from blood, a manufacturing method. When a blood sample and a body fluid sample are respectively injected into the inlet of the device by the method described below, viable CTCs can be isolated and used for the development of specific cancer cell lines. The device has two inlets with a radius of 10 mm or less, a two-loop helical microchannel having a uniform height of a radial inner portion and a radial outer portion, and a rectangular cross-section in which the width of the upper portion is equal to the width of the base, and the two-loop helical microchannel is branched from the CTC and two outlets through which blood cells are separately discharged. The present invention can provide a spiral microfluidic device and module for CTC isolation, a manufacturing method, which can lead to the development of a reported specific cell line by making it possible to isolate viable CTCs by a spiral microfluidic device for CTC isolation derive an effect.

### CROSS-REFERENCE TO RELATED APPLICATION

This application claims priority to Korean Patent Application No. 10-2021-0158829 filed in the Korean Intellectual Property Office on Nov. 17, 2021, the disclosure of which is incorporated by reference herein in its entirety.

### TECHNICAL FIELD

The present invention relates to separating circulating tumor cells (hereinafter CTC) using a spiral microfluid. More specifically, it is about a spiral microfluidic device and module used for CTC separation from blood using inertia and Dean drag, and a manufacturing method therefor.

### DISCUSSION OF RELATED ART

Circulating tumor cells are found in the peripheral blood of cancer patients with a frequency of 1 cancer cell per 10,000,000 (10 million) white blood cells. The probability that CTCs are found in peripheral blood is very low, so a reliable method for detecting and isolating circulating tumor cells is required. Real-time liquid biopsy of circulating tumor cells (CTCs) can improve our understanding of cancer metastasis, tumor growth, heterogeneity and resistance to cancer therapy through further analysis of cells.

Therefore, there is a need for a powerful cell isolation technology that enables rapid and efficient isolation of circulating tumor cells for downstream analysis. On the other hand, the CellSearch system (Janssen Diagnostics) for conventional CTC isolation was approved by the US Food and Drug Administration (FDA) as a CTC isolation and counting product in 2004.

Although CellSearch has limitations with respect to methodology, physics, statistics, and inter-operator variability, it is still used in clinical settings, and improved medical devices in this field have not been clinically approved in the past decade. Although other methods can be applied for CTC detection, most remain in the candidate group, which do not have sufficient ability to process a large amount of blood.

Mass blood processing equipment such as flow cytometry and fluorescence scanning microscopy were also used for CTC isolation. Such techniques require simple and readily available tools to work, but they have limitations in losing rare cells and impairing viability.

Existing size-based filtration methods, such as ‘Isolation by Size of Epithelial Tumor (ISET)’ cells, were also used to isolate CTCs using the size difference between cancer cells and blood cells. However, despite the ease of operation and low cost, problems such as clogging of the filter of ISET and the high frequency of damage to isolated cells may still occur. Mature red blood cells have distinct biological or physical properties that allow them to be easily removed from the blood by gradient centrifugation and lysis. However, some leukocytes may share similar properties with CTCs, and may survive while compromising the purity of isolated CTCs in concentrated samples.

To overcome this limitation, methods that negatively deplete blood cells, such as EPISPOT32 (CHU and UKE) or negCTC-iChip (MGH), have been demonstrated to isolate CTCs in an unbiased manner. In addition, the label-free approach has prompted the development of label-free microfluidic CTC separation techniques based on biophysical properties such as size, density, deformability, or genetic properties, an ongoing problem facing users of affinity binding technology.

However, many of these label-free microfluidic approaches have limitations that prevent widespread adoption. For example, they are often reported to have low throughput and other shortcomings such as clogging, inadequate recovery, complex integration of external force fields, and potential loss of cell viability, especially in clinical settings.

### SUMMARY

The present invention is to make it possible to isolate viable CTCs, being derived from the technical background mentioned above, which was created to provide a spiral microfluidic device and module for CTC isolation that can lead to the development of the reported specific cell line and a method for manufacturing the same. In addition, it is an object of the present invention to provide a spiral microfluidic device and module for CTC isolation showing high recovery, viability, and depletion of WBC by minimizing technical limitations, as well as a manufacturing method.

Another object of the present invention is to provide a spiral microfluidic device, module for CTC separation, and a manufacturing method, which can process larger volumes of blood and give a continuous collection of viable CTCs, facilitating subsequent in vitro CTC culture. Also, we can provide a spiral microfluidic device and module for CTC isolation that can return all blood fractions required for biomarker studies such as plasma, CTCs and peripheral blood mononuclear cells (PBMC), and a manufacturing method.

The present invention for achieving the above object includes the following configuration.

The spiral microfluidic device for CTC separation according to an embodiment of the present invention includes the following devices: a blood sample requiring CTC separation; two injection holes with a radius of 10 mm or less through which samples are respectively injected; a two-loop spiral microchannel having a rectangular cross section in which the radial inner and outer portions are uniform in height and the width of the upper portion is equal to the width of the base; two outlets branching from the two-loop spiral microchannel to separate and discharge CTCs and blood cells.

Meanwhile, the method of manufacturing a spiral microfluidic module for CTC separation according to an embodiment includes the following components and steps: two inlets with a radius of 10 mm or less using standard UV lithography on a silicon wafer; a two-loop spiral microchannel having a rectangular cross section in which the radial inner portion and the radial outer portion are uniform in height and the width of the upper portion is equal to the width of the base; patterning the shape of a spiral microfluidic device for CTC separation, which branches from the two-loop spiral microchannel and includes two outlets through which CTCs and blood cells are separated and discharged; reactive ion etching to form channels in the wafer; trichloro(1H,1H,2H,2H-perfluorooctyl) silanization treatment for a predetermined time to promote mold relaxation; curing polydimethylsiloxane (PDMS) prepolymer after silanization; separating the cured polydimethylsiloxane (PDMS) from the mold; punching holes for inlet and outlet in the separated polydimethylsiloxane (PDMS).

The present technology can provide a spiral microfluidic device and module for CTC isolation, a manufacturing method, which can lead to the development of the reported specific cell line by making it possible to isolate viable CTCs. In addition, it can provide a spiral microfluidic device and module for CTC isolation, a manufacturing method, showing high recovery, viability and depletion of WBCs by minimizing technical limitations.

In addition, it could yield a spiral microfluidic device and module for CTC isolation. This manufacturing method can handle larger volumes of blood, providing a continuous collection of viable CTCs to facilitate subsequent in vitro CTC culture. It could also provide a spiral microfluidic device and module for CTC isolation and a manufacturing method to return all blood fractions required for biomarker studies such as plasma, CTCs, and peripheral blood mononuclear cells (PBMC).

Furthermore, it could provide the following properties for cancer cell isolation: high separation resolution; the processing of isolated cells with high purity; processing capacity to process larger sample volumes; versatility to handle different types of cells; operational robustness; and optimal technology for microfluidic cell separation, etc. Furthermore, it could recover more than 85% of spike cells among cancer cells and destroy 99.99% of white blood cells in whole blood.

### BRIEF DESCRIPTION OF THE DRAWINGS

FIG. 1 is a conceptual diagram of a CTC separation process using a spiral microfluidic device for CTC separation according to an embodiment of the present invention.

FIG. 2 is an exemplary diagram for explaining the operating principle of particle movement in linear and curved microchannels according to an embodiment of the present invention.

FIGS. 3, 4, 5, 6, 7, and 8 are exemplary diagrams for explaining the design and manufacturing method of a spiral microfluidic module for CTC separation according to an embodiment of the present invention.

FIGS. 9, 10, and 11 are exemplary views for explaining the characterization of the spiral microfluidic module for CTC separation.

FIG. 12 is an exemplary view showing the immunostaining state of CTCs concentrated in a clinical patient blood sample.

FIG. 13 is an exemplary diagram for explaining a verification result of a spiral microfluidic module for clinical analysis.

### DETAILED DESCRIPTION

It should be noted that the technical terms used in the present invention are only used to describe specific embodiments, and are not intended to limit the present invention. In addition, the technical terms used in the present invention should be interpreted as meanings generally understood by those of ordinary skill in the art to which the present invention pertains, unless otherwise defined in the present invention. Therefore, it should not be interpreted in an overly comprehensive sense or in an overly narrow sense.

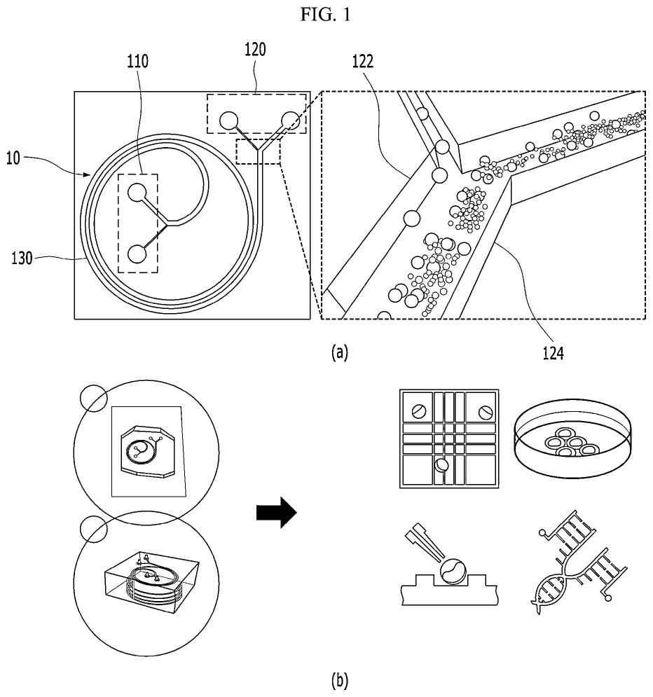

Hereinafter, the present document will describe in detail a preferred embodiment according to the present invention with reference to the accompanying drawings. FIG. 1 is a conceptual diagram of a CTC separation process using a spiral microfluidic device for CTC separation according to an embodiment of the present invention. The spiral microfluidic device (10) for CTC separation according to an embodiment provides an ultra-high throughput-based separation method for CTC isolation, separation and collection from blood. This separation method can quickly and continuously separate viable CTCs by utilizing the distinct focal position of larger CTCs, except for smaller blood cells resulting from inertia and Dean drag combined in spiral microfluidic device 10 for CTC separation according to an embodiment. The simplicity and robustness of device operation can ensure that CTC separation methods can be used in clinical environments with high throughput.

Thus, it minimizes technical limitations and results from high CTC calculation, CTC viability, and WBC depletion. Using the spiral microfluidic device (10) for CTC separation according to an embodiment makes it possible to derive detection sensitivity close to 100% in isolating and detecting CTCs from blood samples of patients with metastatic lung cancer and breast cancer.

Specifically, FIG. 1(a) is a schematic diagram of CTC enrichment by helical channels. As shown in FIG. 1(a), the spiral microfluidic device (10) for CTC separation according to an embodiment includes the following items: a blood sample requiring CTC separation and two inlets having a radius of 10 mm or less into which the sample is injected, respectively (110); a two-loop spiral microchannel (130) having a rectangular cross-section in which the radially inner portion and the outer portion are uniform in height and the width of the upper part is equal to the width of the base; two outlets (120) branched from the two-loop spiral microchannel (130) and separately discharged CTCs and blood cells respectively.

FIG. 1(a) shows that CTCs are concentrated near the inner wall (122), whereas leukocytes, erythrocytes and platelets are concentrated closer to the outer wall (124) of the outlet due to the combined effect of inertial lift and Dean drag forces. As a result, of the two outlets (120) , CTC may be discharged through one outlet on the inner wall side, and white blood cells, red blood cells, and platelets may be discharged through the other outlet on the outer wall side. FIG. 1(b) also describes optical images of single and multi-helical biochips for high-throughput isolation of CTCs from lysed blood and possible downstream techniques for functional characterization of isolated CTCs using helical biochips.

The spiral microfluidic device (10) for CTC separation according to an embodiment can process a larger amount of blood than a conventional microfluidic system, and provides continuous collection of viable CTCs, which may facilitate subsequent in vitro CTC culture. An important advantage of the chip is that it demonstrates the potential ability to return all blood fractions required for biomarker studies such as plasma, CTCs, and peripheral blood mononuclear cells (PBMCs).

Although 10 to 10,000 WBCs per ml of blood (median of 30 samples=3,109 WBCs per ml) remain after spiral chip treatment, the purity of the concentrated CTC samples is enough to perform the downstream sequencing or fluorescence in situ hybridization (FISH) assays. Suffice. Therefore, it is important to isolate more CTCs.

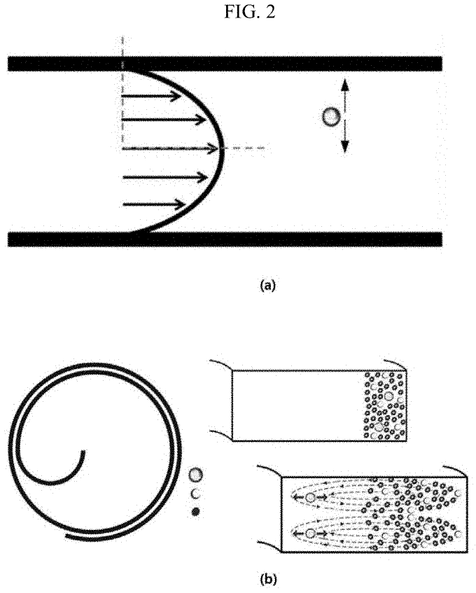

FIG. 2(a) describes the principle of operation according to particle movement in straight and curved microchannels according to an embodiment of the present invention. FIG. 2(a) shows that particles in a rectangular straight channel experience hydrodynamic forces, i.e., shear-induced lift (FIL) and wall-induced lift (FWL), and are concentrated along the perimeter of the channel.

FIG. 2(a) explains that large particles are stabilized closer to the channel center due to the inertial effect of the flow. It also shows that the addition of curvature introduces a secondary cross-sectional flow field perpendicular to the primary flow direction (Dean flow).

Thus, it is confirmed that particles in the spiral channel can move across the main streamline along with the secondary vortex.

Since the channel dimensions affect the equilibrium position of particles and cells, a spiral channel can be designed so that only the larger target particles and cells are concentrated near the inner wall. In comparison, the smaller unwanted particles and cells are dispersed and follow a streamline. As a result, in the channel outlet region, larger particles are concentrated and aligned near the inner wall, while smaller particles occupying a lateral position near the outer wall can be properly achieved. Thus, a spiral microchannel with appropriate dimensions can be designed as shown in FIG. 2(b) to separate larger CTCs and smaller blood cells. FIG. 2(b) shows the working principle of CTC enrichment by a spiral channel of rectangular cross section.

As can be seen in FIG. 2(b), the blood sample is pumped to the device external inlet, while the sheath fluid is passed through the internal inlet.

CTCs stay concentrated near the inner wall due to the balance of inertial lift force (FL) and Dean drag force (FD) at the outlet, whereas blood cells (leukocytes, red blood cells, platelets) migrate along the Dean vortex and exit the device through the outer outlet comes out. The microchannel design parameters can be easily adjusted by changing the size cutoff (the size at which particles and cells are concentrated and the size at which they do not) according to the analytical model described below. The magnitude of the Dean vortex in the curved microchannel is quantified by the dimensionless parameter Dean number (De) given as;

$$De = \frac{ρ {U}_{F} {D}_{H}}{µ} \sqrt{\frac{{D}_{H}}{2 {R}_{c}}} = Re \sqrt{\frac{{D}_{H}}{2 {R}_{C}}} \qquad [ Equation 1 ]$$

where ρ is the fluid density, UF is the average flow velocity, μ is the viscosity of the fluid, RC is the path of the curvature radius channel, DH is the channel mathematical diameter, and Re is the flow Reynolds number (ratio of inertial force to viscous force).

The Dean flows create drag forces on the particles and cells that accompany and drive them along the direction of flow within the vortex. This movement is interpreted as particles moving back and forth across the channel width between the inner and outer walls with increasing downstream distance when visualized from above or below. The speed at which these cells move laterally when flowing through the channel depends on the Dean number and can be calculated as follows.

U_{Dean}=1.8×10⁻⁴De^{1.63} [Equation 2]

The lateral distance a particle traverses along the Dean vortex can be defined as the ‘Dean cycle.’ For example, particles initially located near the outer wall of the microchannel and then moving downstream to the inner wall of the channel at a given distance are considered to have completed half of the Dean cycle. Then the same particle moves back to the outer wall, completing 1 Dean cycle.

Therefore, particles can undergo multiple Dean cycle migration for a given microchannel length as the flow rate (Re) condition increases. The distance of 1 Dean cycle migration can be calculated as follows.

L_{DC}=2w+h [Equation 3]

where w is the width of the microchannel and h is defined as the microchannel height. As a result, the total microchannel length (Lc) required for Dean migration is given as follows.

$${L}_{C} = \frac{{U}_{f}}{{U}_{Dean}} {L}_{DC} \qquad [ Equation 4 ]$$

In this case, the magnitude of the Dean drag is given by Stoke's law.

F_{D}=3πμU_{Dean}a_{c} [Equation 5]

where a_{c} is the cell diameter. Apart from the Dean drag forces, larger particles with diameters comparable to the microchannel dimensions experience significant inertial lift forces (FL) (shear and wall induced), causing focus and equilibrium.

The parabolic velocity profile of the Poiseuille flow results in a shear-induced inertial lift force acting on the particle, guiding the particle from the microchannel center towards the channel wall.

If these particles suddenly appear around the wall as they move closer to the channel wall, they disrupt the rotational wake formed around the particle, inducing lift and moving away from the vessel wall to the center of the microchannel. As a result of these two opposing lift forces, the particle achieves equilibrium (focus) around the microchannel perimeter at distinct and predictable locations. This effect is dominant for particles with sizes comparable to the microchannel dimension ac/h>0.07.

The size of FL is given by

[Equation6]

where CL is the lift coefficient as a function of particle position across the channel cross-section, assuming an average value of 0.5, and G is the shear rate of the fluid. The average value of G for Poiseuille flow is given as G=Umax/DH, where Umax is the maximum fluid velocity and has a close approximation to 2×UF.

$${F}_{L} = \frac{2 ρ {U}_{F}^{2} {a}_{C}^{4}}{{D}_{H}^{2}} \qquad [ Equation 7 ]$$

Thus, the interaction between the inertial lift force (FL) and the Dean drag force (FD) in the curved microchannel reduces the equilibrium positions near the inner channel wall to just two within the upper and lower Dean vortices, respectively.

The two equilibrium positions superimpose each other along with the microchannel height and are equidistant from the microchannel inner wall for given particle size. That is, it can be viewed as a single location across the microchannel width.

These two phenomena (e.g., Dean vortex and inertial focus) can be utilized to separate particle and cell mixtures of different sizes. According to an embodiment, the spiral microfluidic device (10) for CTC separation can separate circulating tumor cells from blood by applying this technique.

The spiral biochip using the spiral microfluidic device (10) according to an embodiment is generally used for separating CTCs with a diameter of 15-20 μm or less and other blood cells [red blood cells (RBCs) and platelets, platelets of 3-8 μm, 10-12 μm of white blood cells)].

According to an embodiment, the spiral microfluidic device 10 for CTC separation may separate CTCs from lysed blood using two inlets, two outlets, and a spiral channel. The basic design elements of the spiral channel of the spiral microfluidic device 10 for CTC isolation according to an embodiment is to include a microfluidic channel with an appropriate channel depth to focus only large target cells near the inner wall. The blood flowing into the device is dispersed by cell size and follows a streamlined shape away from the inner wall.

In one aspect of the present invention, the two-loop spiral microchannel has a cross-sectional width of 450 μm to 550 μm and a radius of curvature of 1 cm or less. Preferably, the cross-sectional width of the two-loop spiral microchannel is 500 μm. At this time, when ac/h meets 0.1 or less, particles can be precisely focused along the inner wall.

Therefore, a spiral CTC isolator operating for separation between CTCs (≥15 μm) and blood cells including RBCs, platelets and WBCs (˜3-12 μm) should have a channel depth of 150 to 180 μm or less. Then, the channel length of the CTC isolator can be determined based on the above-mentioned Equation 4 at a Raynolds number of 20 to 100 or less where the inertial focus of large particles generally occurs. Also, the inlet partition of the CTC isolator was designed to be 75 μm, and the sample inlet near the outer wall and the sheath inlet near the inner wall were designed to be 425 μm. Therefore, if the sample entrance is small, all cells can enter the spiral channel and start lateral movement in a similar position. The segmentation design of the channel outlet can be determined by studying the lateral position of particles and cells across the channel width at various flow rates.

Briefly, large 15 μm diameter particles (representing CTC) first gather near the inner wall, and the inner wall and small 6 μm diameter particles (representing RBC) identify the set of bonds required to travel one complete dean cycle, which consists of a flow rate range and a flow rate and channel length.

We measure the particle distribution across the channel width at various input cell concentrations in a window of flow rate and channel length that meets both requirements. In addition, the optimal position of the outlet division is checked so that the width for the CTC collection outlet near the inner wall is 150 μm and the width for the waste outlet near the outer wall is 350 μm.

First, we design a spiral system (i.e., a two-step cascaded system) to isolate high-purity CTCs from whole blood, demonstrating that the cascaded spiral biochip can process blood with a hematocrit of 20-25% in ˜3 ml Rare cells can be processed and concentrated at a /h rate (75 ml for 150 min). However, the protocol can be modified to include an RBC lysis step for improved yield and target cell purity to increase the throughput of the system while simplifying operation and automation.

Nucleated cells generated by RBC lysis were resuspended in saline before helical treatment to 0.5 times the original whole blood volume (2-fold concentration, ^{˜}14×10 ⁶ nucleated cells per ml). This RBC lysis pretreatment step substantially reduces the number of off-target blood cells in the sample.

Thus, unwanted cell dispersion due to cell-cell interaction can be mitigated (7.5 ml for 37.5 min for a single chip). In addition, the throughput of spiral cell sorting can be further improved by stacking three spiral microfluidic devices (10) for the separation of CTCs to build a multi-system type, that is, a spiral microfluidic module for separation of CTCs.

The 3× multisystem, or spiral microfluidic module for CTC isolation, has a common inlet and outlet of three spiral microfluidic devices operating in parallel with each other.

The spiral microfluidic module for CTC isolation operates at a higher throughput, allowing it to process a larger blood volume (7.5 ml for 12.5 min for the multiplexing chip).

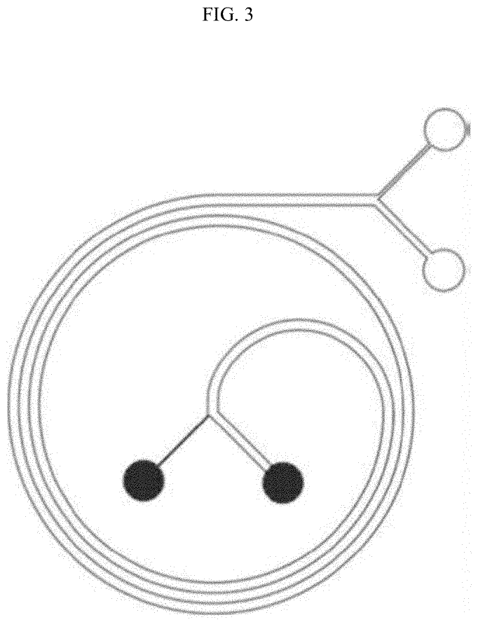

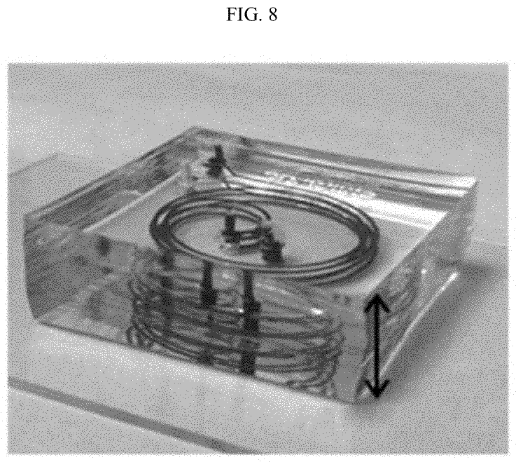

Hereinafter, the design and manufacturing method of the spiral microfluidic module for CTC separation will be described. FIG. 3 or FIG. 8 is an exemplary diagram for explaining a spiral microfluidic module's design and manufacturing method for CTC separation according to an embodiment of the present invention.

Specifically, FIG. 3 is an exemplary CAD drawing of a spiral microfluidic device included in a spiral microfluidic module for CTC separation according to an embodiment.

As shown in FIG. 3, the spiral microfluidic device design is implemented to consist of a two-loop spiral microchannel with two inlets and two outlets with a radius of 10 mm or less.

Preferably, the width of the two-loop spiral microchannel cross-section is 500 μm, and the outlet branch can be optimized to have two outlet diameters of 150-350 μm, respectively.

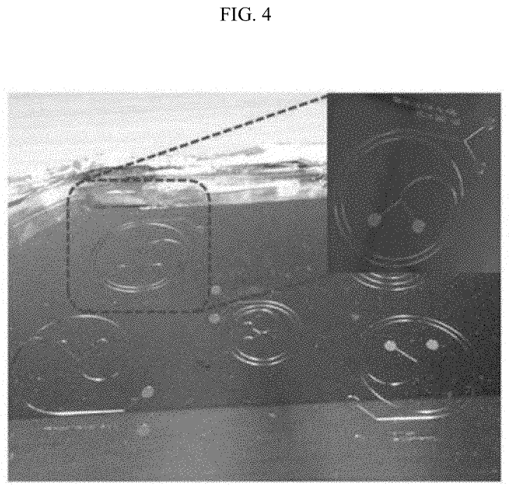

FIG. 4 is an exemplary diagram of the mold manufacturing process using standard micromachining techniques.

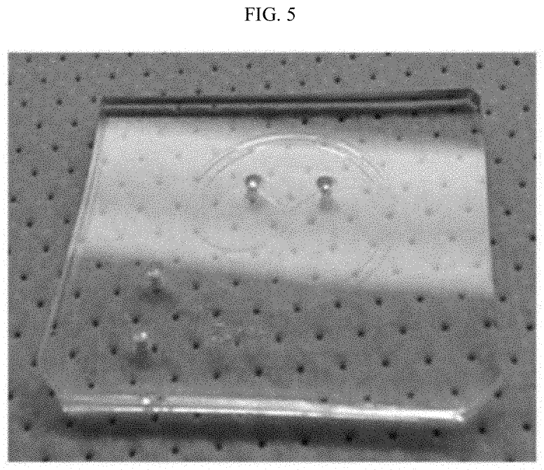

FIG. 5 shows the device diagram, showing soft lithography and pattern transfer into a single layer of polydimethylsiloxane (PDMS) using the fabricated mold, and fluid access to the inlet and outlet. At this time, a precision punch is used to penetrate the device.

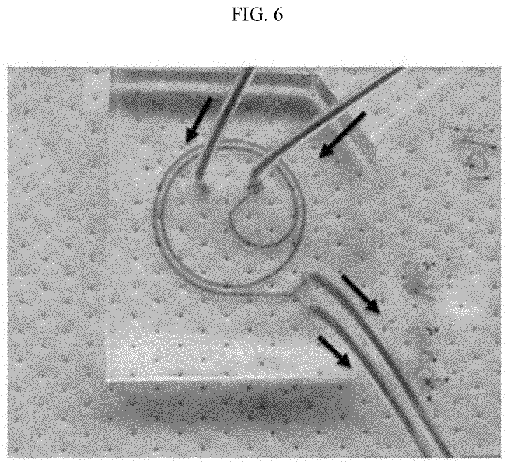

FIG. 6 is an exemplary view of the completed spiral device after bonding to the glass slide. Red food coloring was used to improve the visualization of the channel.

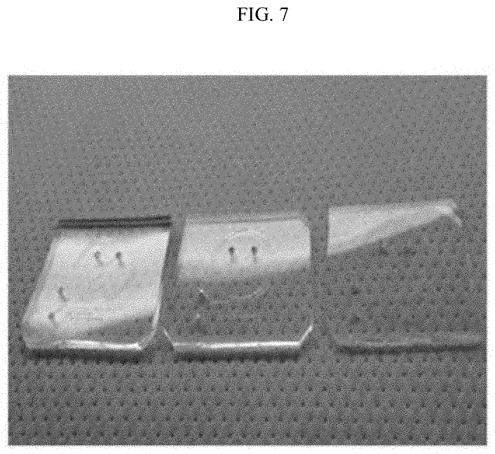

Also, FIG. 7 shows three individual PDMS replicas with pierced fluidic access.

FIG. 8 is an exemplary diagram of a helical microfluidic module for CTC separation obtained by stacking three individual polydimethylsiloxanes (PDMS) together using plasma bonding and passive alignment.

A silicon master of microfluidic channels can be fabricated using standard microfabrication techniques and used to produce spiral microchips, i.e., spiral microfluidic modules, as previously described. Briefly, a 6 inch diameter silicon wafer is first patterned using standard UV lithography and etched using deep reactive ion etching (DRIE) to form channels (˜170 μm etch depth) in the wafer.

After etching, the patterned silicon wafer (FIG. 3b) was cleaned using acetone and isopropanol and treated with (1H,1H,2H,2H-perfluorooctyl) for 2 h to promote relaxation of the polydimethylsiloxane (PDMS) mold.

Experimental Design

The design of the spiral microfluidic device for CTC isolation according to an embodiment consists of a two-loop spiral microchannel with two inlets and two outlets with a radius of ˜10 mm.

At this time, as shown in FIG. 3, the width of the channel cross section is 500 μm and the outlet branch can be optimized to 150-350 μm respectively.

Thus, a silicon master of the microfluidic channel is fabricated using standard microfabrication techniques and used to produce spiral microchips, or spiral microfluidic modules.

Briefly, a 6 inch diameter silicon wafer is first patterned using standard UV lithography and etched using deep reactive ion etching (DRIE) to form channels (˜170 μm etch depth) in the wafer.

After etching, the patterned silicon wafer as shown in FIG. 4 was cleaned using acetone and isopropanol and treated with trichloro(1H,1H,2H,2H-perfluorooctyl)silane for 2 hours to promote relaxation of the polydimethylsiloxane (PDMS) mold.

After silanization, the PDMS prepolymer is mixed with a curing agent in a ratio of 10:1 (wt/wt) and poured onto the silanized wafer. Wafers are baked at 80° C. for 1-2 hours.

After curing, peel the PDMS from the mold and punch access holes (1.5 mm) for fluid inlet and outlet as shown in FIG. 5.At this time, using a Uni-Core puncher (Sigma-Aldrich) and using an oxygen plasma machine to complete the channel, the PDMS device is irreversibly coupled to a fine glass slide as shown in FIG. 6.

Afterwards, three PDMS molds were fabricated as shown in FIG. 7 and laminated together using plasma bonding and manual alignment to fabricate a multiplexing device, that is, a spiral microfluidic module for CTC separation.

Finally, as shown in FIG. 8, the spiral microfluidic module was irreversibly bonded to the glass slide using an oxygen plasma machine and placed inside an oven at 70° C. for 30 min to further strengthen the bonding. Then, the chips can be primed to prepare them for use.

Meanwhile, characterization using surrogate microbeads and cell lines may be performed.

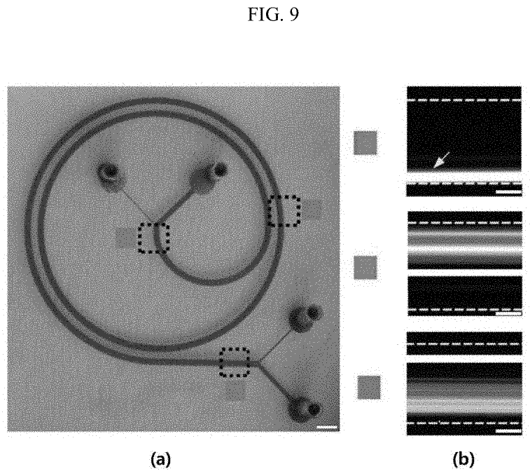

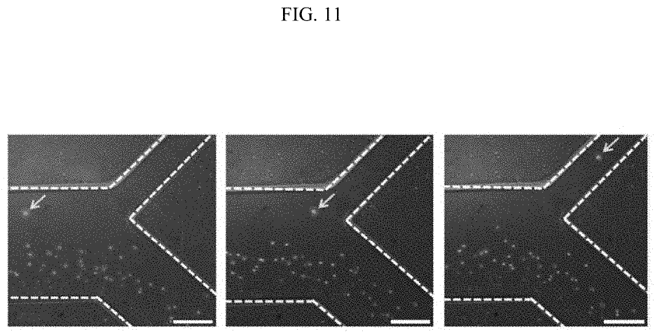

FIG. 9 and FIG. 11 are exemplary views for explaining the characterization of the spiral microfluidic module for CTC separation. Specifically, FIG. 9(a) is a photograph of a spiral micro-indwelling device developed for CTC separation, filled with blue dye to visualize microchannels.

The scale bar in FIG. 9(b) shows the verification of the design principle using fluorescently labeled polystyrene particles. Overlaid images showing the distribution and location of 6 and 15 μm particles at the inlet (X), channel middle (Y), and outlet (Z) are observed in the figure.

The particles randomly distributed at the inlet can be collected separately at the next outlet after forming an ordered concentrated stream.

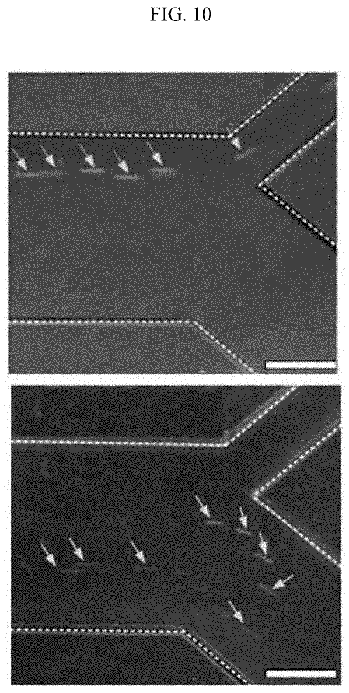

FIG. 10 is a top, average composite image showing the focal position of MCF-7 cells at the exit of the spiral device.

The lower part showing the focal position of leukocytes at the exit of the spiral micro-indwelling device is an artificially synthesized image, and the scale bar indicates units of 200 μm.

FIG. 11 is a time-sequential image showing the separation of CTCs from lysed blood using a spiral microfluidic module. The dotted lines indicate the boundaries of the microfluidic channels in all panels, and t(time)

An important aspect of the optimization process of the spiral microfluidic device for CTC separation according to an embodiment is to characterize the behavior of particles or cells in flow within the designed cell sorter. As described above, the optimal channel depth and outlet partition design and operating flow are finally determined based on actual experimental results.

To save cost and time, initial device characterization was usually performed with dummy particles as suggested in step 27 of the procedure. To mimic blood components, 6, 10, and 15 μm dummy particles representing RBC, WBC and CTC, respectively, were used. In addition, to verify the results generated in the particle study, it was confirmed as an actual cancer cell line in the newly synthesized optimized system as shown in FIG. 9.

Device characterization using cell samples is ideal for identifying discrepancies in hydrodynamic behavior between rigid microbeads and deformable cancer cells.

As shown in FIG. 10, cancer cells of lysed blood (WBC) or cell lines were individually flowed into the optimized device and cell distribution across the channel width was observed in the exit region.

In this case, a microscope equipped with a phase-contrast light source and a high-speed camera is used. Therefore, using a microscope equipped with a phase-contrast light source and a high-speed camera, observation of cell distribution across the channel width in the two outlet regions for cancer cells of lysed blood (WBC) or cell lines flowing individually into the spiral microfluidic device can be confirmed.

Overall, channel dimensions optimized based on particle results should work for real cell samples. However, the optimal working flow rate is slightly different because the interaction between the fluid and the deformable cell results in additional lift forces.

These additional forces affect the exact equilibrium position of the cell within the channel cross-section. The distribution of particles or cells across the channel width can be observed at different locations along the channel length at various flow rates.

For consistency, the effect of the total flow rate on particle and cell behavior was studied by fixing the sample input flow rate at 100 μl/min and changing the coating buffer flow rate.

For larger (>15 μm) particles and cells, we mainly investigated the exit region of the spiral channel, since the larger particles and cells do not move much laterally when they reach an equilibrium position near the inner wall.

For smaller particles and cells, the behavior along the channel length mediated by lateral Dean migration can be monitored.

Optimally, smaller particles and cells migrate first from the outer wall side to the inner wall side of the inlet region, and then move again along the length of the channel. They then have to move back to the area closer to the outer wall with some scattering (i.e., go through a complete Dean cycle).

Adjustment of the envelope buffer flow rate (and hence the total flow rate) can alter the number of Dean cycles that small particles and cells take within a channel of a given length.

Isolation of spike cell lines from lysed blood samples is possible. In real samples, CTCs are found infrequently, with blood cells such as RBCs (billions per ml), platelets (millions per ml), and WBCs (millions per ml) constituting 99.99% of the total cell count in clinical whole blood samples.

Red blood cells, leukocytes, and hemoglobin present with the target cancer cells will affect cell concentration and flow within the spiral channel. Conventional RBC lysis technology (using ammonium chloride solution) increases throughput while reducing the number of cellular components flowing into the spiral biochip. Although WBCs make up only 1% of the total blood volume, it is still difficult to efficiently isolate CTCs from them. An extensive review of the previously proposed methodology for increasing the leukocyte depletion capacity of the spiral biochip to isolate CTCs was performed. Removal of most of the RBCs by lysis allows the sample to be processed without dilution (increased throughput) and, as shown in FIG. 11, allows better separation of larger cancer cells from smaller blood cells due to reduced cell concentration.

Moreover, this additional pretreatment step does not impair cancer cell recovery and does not alter the viability and morphology of the recovered cells. The presence of blood cells along with the target cancer cells will affect cell concentration and flow within the helix channel.

To increase throughput while reducing the amount of cellular components flowing into the spiral biochip, a conventional RBC lysis technique (using ammonium chloride solution) is used.

To test the performance of the spiral biochip for isolation and repair of CTCs, commercially available cancer cell lines can be used to characterize the biochip. Different cell lines must be used because they exhibit a wide range of cell sizes, so that the device can be optimized for the isolation of CTCs of different cancer types. By confirming a high degree of recovery (˜85%) across multiple cell lines for clinically relevant spiking doses, we demonstrated the ability to successfully classify spiked cancer cells from blood components.

The purity of the concentrated sample is critical in many downstream molecular analyzes where leukocyte contaminants can significantly lower the signal-to-noise ratio, leading to inaccurate diagnoses. Starting with an initial concentration of nucleated cells at a level of ˜14×10⁶ (7.5 ml of blood is dissolved and resuspended in 3.75 ml of PBS), the spiral microfluidic module according to one embodiment can deplete ˜99.99% of leukocytes in a healthy sample, thus providing a purer CTC fraction at the outlet.

However, the capture purity of a clinical sample depends on a function composed of several variables, including the number of isolated CTCs, the number of contaminated WBCs that vary from patient to patient depending on the type of cancer, blood quality, and stage of the cancer (i.e., some patients have high numbers of white blood cells in their blood due to chemotherapy). Therefore, it is difficult to say uniformly about the purity of the sample, and the value may be different for each sample.

Application of the Protocol

The spiral microfluidic module according to an embodiment may be useful for both research and clinical applications. CTC quantification can provide insight into cancer progression, therapeutic efficacy and survival prognosis.

It was firstly reported that patients characterized by 5 CTCs per 7.5 ml of peripheral blood had a lower overall survival rate by Cristofanilli et al., which was supported by Hayes et al. who studied the prognostic nature of CTC.

Therefore, CTC enumeration may have the most important clinical significance in guiding individualized treatment decisions.

A research on CTCs can also be an asset to research that sheds greater light on cancer biology.

CTC interrogation can be performed using DNA or RNA-FISH (Box 1) or genetic analysis techniques. The immune properties of CTCs at various cancer stages, types and locations can represent important diagnostic-related differences.

CTC culture can also be used for drug testing on various platforms such as 3D microfluidic systems. Recently, social and academic interest in cancer genome research is increasing.

Genome sequencing and mapping of the listed CTCs can be performed to identify regions of DNA or RNA common to malignant cancers, thereby facilitating drug discovery.

In addition, nucleotides can be transcribed and translated into proteins to understand how cancer cells interact and manipulate their microenvironment, either directly or through bioinformatics tools (e.g., European Institute of Molecular Biology (EMBL) Nucleotide Sequence Database). And that information could complement the findings of acellular genetic studies.

The spiral biochip, that is, the spiral microfluidic module according to an embodiment, has three characteristics that add value to clinical and research goals.

First, the listed CTCs are quiescent and not immobilized on the chip, facilitating immediate downstream manipulation and analysis such as culture.

Second, the method of CTC isolation is independent of tumor antigen. Thus, the platform could potentially be more sensitive than a competing immune-based platform. In addition, CTC clusters believed to be associated with tumor metastasis can be searched for in blood samples.

Third, it shows high purity in the concentrated sample with up to 4 log WBC consumption.

In other words, the high specificity of the CTC isolation technique according to an embodiment has the effect of enabling higher accuracy during single cell analysis as well as genome sequencing and mapping. More specifically, the design of the spiral microfluidic module and the mold preparation procedure can be first drawn in the microfluidic design in AutoCAD software as shown in FIG. 3.

Then, for mask printing, you provide the design (usually an AutoCAD file) to the vendor to be masked. Alternatively, the master mold can be made using conventional micro-milling on aluminum or stainless steel.

The advantage of this method is that it does not require silanization of the master mold for biochip cloning and can be reused for a long period of time.

After that, a photolithography process is performed in a clean room to fabricate a spiral biochip master (photolithography).Specifically, 6 ml or less of AZ9260 photoresist is dispensed on a 4 inch wafer that has been washed and dehydrated.

First, the AZ9260 film distributed on the wafer is uniformly spin coated for 10 seconds by using a spinner by ramping from 100 rpm/s to 500 rpm, and then ramping to 3000 rpm at 500 rpm/s acceleration for 30 seconds. Then, pre-bake the coated wafer to a flat state at 110° C. for 5 min.

Then, using a functional printed transparent film mask, the wafer is exposed to the appropriate amount of UV light to create the master.

Wafers exposed to photoresist developer (AZ400K) are then developed for 5 minutes (1:4 dilution), the substrates are rinsed thoroughly with deionized water, and then dried with a gentle stream of pressurized nitrogen gas.

Next, etch a silicon wafer of ˜165±5 μm using a DRIE machine. Accurate etch depth is important for optimal performance of spiral biochips. When the channel depth is <160 μm or >170 μm, the particle focus is compromised and the concentrated sample is highly contaminated.

After then, the silicon wafer is silanized using 150 ml of trichloro(1H,2H,2H,2H-perfluorooctyl)silane for 1.5 hours using a vacuum dryer.

The silanized wafer master can then be stored under a cover to prevent long-term dust exposure and reused for further device fabrication using soft lithography.

The spiral biochip production (soft lithography) process can be performed outside a clean room in a laboratory environment. Specifically, the PDMS base and the PDMS curing agent are uniformly mixed in a weight ratio of 10:1.

While holding the silane-treated wafer master (using double-sided adhesive) in the center of a 15 cm diameter Petri dish, pour 77 g of the PDMS mixture into the dish.

Then, degas the PDMS in a vacuum desiccator for 1 h, and when there are no air bubbles in the PDMS, bake the dish with the master at 70-80° C. inside the oven for at least 2 h to cure the PDMS.

Cut and peel the cured PDMS from the master. At this time, measure the height of the cured PDMS device for the first casting to ensure that the channel height of the fabricated device is within tolerance (i.e., 1-5 μm).

Cured PDMS can be stored for a long time in a clean environment. Holes are subsequently drilled to make the inlet and outlet of the device at the appropriate points for each piece of PDMS. For single-layer spiral chips, use a 1.5 mm diameter puncher. To increase throughput with multiple devices at multiple levels, a 4 mm diameter puncher is used for the bottom layer and a 1.5 mm diameter puncher is used for the top layer.

Clean all PDMS surfaces and glass slides with scotch tape by tapping gently afterwards.

Check that the entire area is in contact with the tape and visually check for dirt.

Then, a glass slide with the surface to be bonded with the characteristic PDMS piece facing up is cleaned using a plasma apparatus.

Bond the feature-length PDMS piece to the glass slide by contacting the bonding surface and press the device.

Gently complete bonding for 30 seconds with tweezers.

Place the bonded PDMS device on an 85° C. hot plate or oven for 30 min to further strengthen the bonding and cool it down for 5 minutes.

First, one-to-one bonding is performed to make the assembled pieces into a two-layer structure. Then, bonding between the assembled pieces can be performed to obtain the final multi-device with the desired spiral channel copies.

After this, connect the tubing to the inlet and outlet of the microfluidic device for chip priming.

And prime the device for 10-15 minutes before running the sample.

A syringe pump is used to control the flow rate and the flow rate video is captured with a microscope and a high-speed camera.

In particular, it is necessary to check that there are no air bubbles or foreign substances that may obstruct the flow of the microfluidic device. In addition, the Retort stand should be placed at the same height in all experiments to ensure pressure consistency.

If sterile conditions (culture, etc.) are required, load the syringe with 70% (vol/vol) ethanol in sterile deionized water, then push the plunger forward to remove any visible air bubbles in the syringe.

Connect the optionally mounted syringe to one of the spiral inlets via a precision syringe tip and a 1.5 mm diameter silicone tube, then insert a separate tube (i.e., equal length (˜15 cm)) into the outlet of the spiral chip to deliver the sample in the collection tube.

Then mount the syringe on the syringe pump and run 70% (vol/vol) ethanol at the appropriate flow rate (750 μl/min for single device) on the spiral chip for 5 min to sterilize the system and remove any air bubbles present inside the system.

After this, load the external buffer (1× sterile PBS with 0.5% (wt/vol) BSA) into a separate 60 mL syringe and run the external buffer through the system for 5 min to coat the surface of the microchannel and remove it from the system. Wash off residual ethanol. In this case, an inverted-image microscope is used, and a bright field mode is applied to inspect the microfluidic channels and to ensure that no air bubbles are present in the channels.

Increasing the Sheath buffer flow rate may help to remove air bubbles.

Quality control (QC) is performed through the device primed with particle suspension (˜3-6 μm diameter particles at 0.1% vol/vol) and external buffer.

Connect the syringe equipped with the particle suspension to the outer inlet of the spiral chip and the syringe equipped with the coating buffer to the inner inlet of the chip.

After mounting both syringes on the pump, proceed to run the particle suspension at a flow rate of 100 μl/min and 750 μl/min through a single spiral until the particle suspension is observed entering the main microchannel.

Meanwhile, to lyse blood, warm the blood sample to room temperature (24 to 26° C.) and add 3× RBC lysis buffer at a 1:3 volume/volume ratio.

Mix by inverting the conical tube and incubate the mixture for 5 min at room temperature on a stirring platform or periodically invert until the color changes to dark red.

If using blood with cancer cells added at this time, spike the desired number of cancer cells prior to RBC lysis as described in the reagent settings.

After incubation, the cells are harvested by centrifugation at 300 g for 5 min at room temperature, and then re-fixed to the cell pellet in 1×PBS at the protocol-optimized concentration (2-fold concentration, ˜14-fold×10⁶ nuclear cells per ml). Then resuspend the pellet by tapping the tube or mixing well with light pipetting, and place the sample on ice. At this time, the hemolyzed blood sample may be stored at 4° C. for 2-4 hours until processed.

Check the spiral exit by adjusting the microscope lens (10× magnification) for hemolysis using single or multiple spiral biochips.

Place sterile 15 ml (for concentrated sample collection) and 50 ml (waste collection) conical tubes at the waste and sample outlet collection site.

Then, connect a 50 ml syringe filled with running buffer to the sheath inlet of the spiral chip.

The flow rate of this pump should be set to 750 μl/min when processing using a single recurve or 2,100 μl/min when processing using a multiplex chip (three curves).

The biochip is primed by operating the sheath buffer at a current flow rate of 750 μl/min for 2 minutes.

Put the sample syringe containing the lysed sample at the desired concentration into the second pump and adjust the flow rate to 100 μl/min.

Connect the syringe to the chip using a Tygon tube and precision tip.

Then, the processing of the sample begins with flowing in the sheath buffer for about 1 minute until the flow is stabilized.

After stabilization, guide the sampling tube to the 15 ml conical tube, start the sample pump, and continue collecting for 10-30 minutes (7.5 ml of lysed blood).

Immunofluorescence staining of the isolated cells is also performed. Cells should be stained prior to counting CTCs. As an alternative method of immunofluorescence staining, cells can be characterized by FISH.

Afterwards, the collected sample is concentrated by centrifugation at 300 g for 3 minutes at room temperature. Then, the excess supernatant is removed and the cells are re-treated up to 250 μl of PBS.

Cells are fixed with 4% (wt/vol) paraformaldehyde at room temperature for 10 minutes.

Then, after washing the fixed cells with 1-2 ml of PBS buffer supplemented with 0.5% (wt/vol) BSA, centrifuge at 300 g for 3 minutes at room temperature. At this time, the fixed cells can be stored overnight in PBS buffer at 4 ° C. After removing the excess supernatant and re-running the cells in ˜250 μl containing 0.1% (vol/vol) Triton X-100, permeabilize the cells at room temperature for 1 to 5 minutes.

After washing the permeabilized cells with 1-2 ml of PBS buffer supplemented with 0.5% (wt/vol) BSA, permeabilize cells at 300 g for 3 minutes at room temperature

Thereafter, the necessary binding antibody is directly added to the cell suspension and cultured on ice for 30 minutes. Use binding antibodies such as FITC-binding pancytoceratin (CK) antibodies (1:100) and APC-binding CD45 antibodies (1:100) to identify positive CTCs. In addition, Hoechstye (1M, 1:1,000) is added to the dye reagent solution for nuclear dyeing.

After washing the stained cells with 1-2 ml of PBS buffer supplemented with 0.5% (wt/vol) BSA, centrifugation is performed at 300 g for 3 minutes at room temperature. Then, the stained cells are resuspended at a concentration of 250 μl or less and transferred to a single well of a 96-well plate. For counting or characterization of cells, cells can be counted using custom imaging platform options or manual options.

Each well is imaged and scanned for imaging and enumeration using the custom platform option. Each well is scanned in a 1 mm×1 mm grid format using Metamorph software. Select the desired particle by selecting the manual threshold function in ImageJ software for exhaustive mapping and enumeration of cells in a single well. On the other hand, the manual counting option of cells acquires images of cells at 40× magnification. Then compare the corresponding image sets.

This identifies Hoechst-positive/pan-CK+/CD45-negative (CD45−) cells with round nuclei and high nuclear-to-cytoplasmic (N/C) ratio. The cells are considered putative CTCs, and their proportions compared to other cell types are determined.

Accordingly, this protocol presents a method to perform a high-throughput method of CTC enrichment at high sensitivity and purity. Characterization of the system using cell lines is an example of the results achievable through this protocol.

In particular, treatment of clinical blood samples from patients with locally advanced or metastatic non-small cell lung cancer (n=15) or breast cancer (n=15) was made. The enriched cells were stained with pan-CK (an epithelial marker composed of CK8, CK9, CK18 and CK19) and CD45 (leukocyte marker) antibodies to confirm the presence of epithelial cancer cells (FIG. 5a).

Pan-CK+/CD45−/Hoechst+ circular cells with a high nuclear to cytoplasmic ratio were listed as putative CTCs.

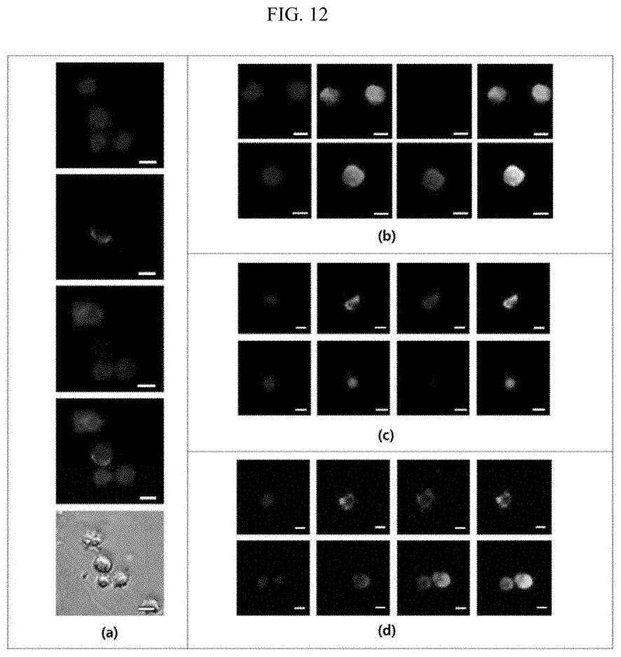

These parameters were adapted from the parameters used for disseminated tumor cell identification. FIG. 12 is an exemplary view showing the immunostaining state of CTCs concentrated in a clinical patient blood sample.

Specifically, FIG. 12(a) shows that the presence of epithelial cancer cells was confirmed by staining the enriched cells with pan-CK (epithelial marker) and CD45 (leukocyte marker) antibodies (1:100, MiltenyiBiotec Asia Pacific). The presence of enriched cells could also explain the heterogeneity of CTCs isolated by label-free techniques, stained with various EMT-associated markers such as EpCAM(b), CD44, CD24(c) and E-cadherin(d).

For comparative analysis between samples in FIG. 12(b) or (d), cell counts were converted to CTC/ml. Further immunostaining with ET-related markers showed a variable in the putative CTC for EpCAM, CD44, CD24 and E-cadherin. expression.This protocol can identify CD44+CD24−Hoechst+ cells that correspond to a subpopulation of breast cancer stem cells. Cancer stem cells are known to cause tumors and exhibit resistance or resistance to certain drug therapies.

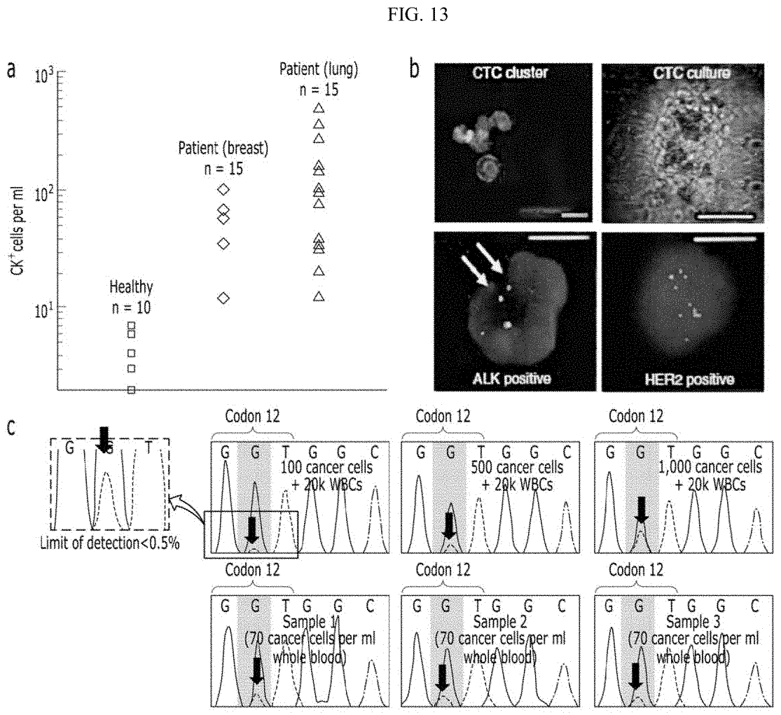

FIG. 13 is an exemplary diagram to explain the verification result of the spiral microfluidic module for clinical analysis. As shown in FIG. 13(a), 10 samples of healthy volunteers were treated as negative controls, and a threshold of >7 CK+ cells can be derived for positive patient samples.

In addition, it is possible to isolate ranges of 12-1,275 CK+ breast CTC and 10-1,535 CK+lung CTC in patient samples that are clearly distinct from those obtained in healthy samples.

On the other hand, as shown in FIG. 13(b), the label-free technique can isolate CTC clusters (microembolisms),which has been reported to correlate with improved survival and proliferation.

CTCs enriched in samples obtained from lung cancer or breast cancer patients in FIG. 13(b) were obtained by amplification of the HER2/neu gene using DNA-FISH (encoding anaplastic lymphoma receptor tyrosine kinase) break-apart probe or DNA-FISH. Rare ALK mutations were marked as positive. In addition, the enriched CTCs are viable, potentially allowing for downstream analysis including culture.

Efforts are underway to further purify the CTC fraction as shown in FIG. 13(c) and to improve the current yield of the helical apparatus for use in delicate downstream analyzes such as sequencing.

Once the microfluidic device is fabricated and primed as described, the spiral biochip is easy to use and can be further manipulated according to specific needs. In a further aspect of the present invention, an anticancer agent reactivity test method using cancer cell mobility analysis applying a CTC sample separated from a spiral microfluidic device according to an embodiment is performed by introducing the anticancer agent into the chamber.

Here, the spiral microfluidic device for CTC separation includes two outlets through which CTCs and blood cells are separately discharged, which has two inlets with a radius of 10 mm or less through which blood samples and epithelial fluid requiring CTC separation are each injected; the radial inner portion and the radial outer portion are uniform in height; a two-loop spiral microchannel having a rectangular cross-section in which the width of the upper part is equal to the width of the base; branched from the two-loop spiral microchannel.

Subsequently, a change in the location of cancer cells in the chamber is periodically measured for a set time period.

In one embodiment of the present invention, for example, the anticancer agents Daunorubicin, Dexamethasone, Doxorubican, Etoposide, and Docetaxel are put into the chamber into which the CTC samples are introduced by classification in a 96-well plate, and the concentration of each anticancer agent is 100 uM (micro mol), 30 uM, 10 uM, 3 uM, 0.3 uM, 0.1 uM, and 0.03 uM, respectively. In addition, in the process of culturing for 24 hours, 48 hours, or 72 hours after administration of the anticancer agent, cytotoxicity and cell motility of liver cancer cells or lung cancer cells or CTC samples to specific agents are observed.

In an embodiment of the present invention, each cancer cell's center of gravity value is calculated using the cell position change measuring means to measure the motility, that is, the amount of movement of the cancer cells.

In one embodiment, the value of the center of gravity of cancer cells is calculated as x, y coordinate values (x, y).That is, by calculating the value of the center of gravity for each cancer cell having a unique identification number, it is possible to calculate the time when measuring the center of gravity of the cancer cell.

Thereafter, by comparing the predetermined time interval, that is, the set time and the elapsed time, the center of gravity value of the cancer cells for each time is calculated periodically using the above-described method, and the movement amount of each cancer cell is measured according to the change of the coordinate value. In this case, the setting of the measurement time interval and the end time (set time) can be variously changed by the user.

As described above, the center of gravity value measured over a certain time interval for each number of cancer cells can be graphed through the output unit, thereby measuring a change in the position of the cancer cell individual.

In this case, the movement distance of the cancer cells can be obtained through the difference between the first measured center of gravity when the cancer cells were first observed and the last measured center of gravity when the cancer cells were last observed. The movement speed of the cancer cells can be easily calculated through the movement distance of the cancer cells according to the movement time of the cancer cells, and the movement direction and speed or acceleration can be measured by using the first measured center of gravity as the relative coordinate origin.

In this way, it is possible to automatically and quickly measure the amount of movement, that is, the change in the amount of movement of cancer cells. In addition, the apoptosis of the liver cancer cell line and lung cancer cell line used for the measurement is assumed to be dead when there is no change in cell migration for a predetermined time, and the death rate can be easily measured as follow;

[Number of cancer cells with zero movements for a predetermined time/Total number of cancer cells]×100

The above-described method may be implemented using an application or implemented in the form of program instructions that may be executed through various computer components and recorded in a computer-readable recording medium. The computer-readable recording medium may include program instructions, data files, and data structures alone or in combination.

The program instructions recorded on the computer-readable recording medium are specially designed and configured for the present invention, and may be known and used by those skilled in the computer software field.

Examples of computer-readable recording media include:

1) magnetic media such as hard disks, floppy disks and magnetic tapes;

2) optical recording media such as CD-ROMs and DVDs;

3) magneto-optical media such as optical disks;

4) hardware devices specially configured to store and execute program instructions, such as ROM, RAM, flash memory, etc.

The program instructions recorded on the computer-readable recording medium are specially designed and configured for the present invention, and may be known and used by those skilled in the computer software field.

Examples of the computer-readable recording medium include 1) magnetic media such as hard disks, 2) magneto-optical media such as floppy disks 2) optical recording media such as CD-ROMs and DVDs, 3) magneto-optical media such as floptical disks, and 4) hardware devices specially configured to store and execute program instructions, such as ROM, RAM, flash memory, etc.

Examples of program instructions include not only machine language codes such as those generated by a compiler, but also high-level language codes that can be executed by a computer using an interpreter or the like. The hardware device may be configured to operate as one or more software modules to perform processing according to the present invention, and vice versa.

Although the above has been described including the embodiments, various modifications and changes can be made to the present invention by those skilled in the art without departing from the spirit and scope of the present invention as set forth in the claims below.

What is claimed is:

1. A spiral microfluidic device for CTC separation with two outlets , comprising:
two inlets, each with a radius of 10 mm or less, into which blood samples and epithelial fluid requiring CTC separation are injected;
a two-loop spiral microchannel having a rectangular cross section in which the radial inner portion and the radial outer portion are uniform in height and the width of the upper portion is equal to the width of the base; and
two outlets branching from the two-loop spiral microchannel to separate and discharge CTCs and blood cells.

2. The spiral microfluidic device of claim 1, wherein
the two-loop spiral microchannel,
has a cross-sectional width of 450 μm to 550 μm and a radius of curvature of 1 cm or less.

3. The spiral microfluidic device of claim 1, wherein
the size of the Dean vortex in the two-loop spiral microchannel is quantified by the dimensionless parameter Dean number (De), wherein
Dean number(De) of spiral microfluid device is calculated by Equation
$$De = \frac{ρ {U}_{F} {D}_{H}}{µ} \sqrt{\frac{{D}_{H}}{2 {R}_{c}}} = Re \sqrt{\frac{{D}_{H}}{2 {R}_{C}}} .$$

4. The spiral microfluidic device of claim 3, wherein
when flowing in the two-loop spiral microchannel of the spiral microfluidic device for CTC isolation, the rate at which the cells move laterally (UDean) is calculated by Equation
U_{Dean}=1.8×10⁻⁴De^{1.63}

5. The spiral microfluidic device of claim 4, wherein
the length of 1 Dean cycle migration of the two-loop spiral microchannel is a spiral microfluidic device for CTC separation calculated by Equation L_{DC}=2w+h

6. The spiral microfluidic device of claim 5, wherein
the total length of the two-loop spiral microchannel required for dean migration is calculated by Equation
$${L}_{C} = \frac{{U}_{f}}{{U}_{Dean}} {L}_{DC} .$$

7. The spiral microfluidic device of claim 1, wherein a spiral microfluidic device for microfluidic module for CTC isolation, obtains the results of observation of cell distribution over the channel width in the two outlet regions for cancer cells of lysed blood (WBC) or cell lines flowing individually from a microscope equipped with a phase contrast light source and high-speed camera to the spiral microfluidic device.

8. The spiral microfluidic device of claim 1,wherein three stages of the spiral microfluidic device for CTC separation of claim 1 are stacked in a spiral microfluidic device for microfluidic module for CTC isolation.

9. The spiral microfluidic device of claim 8,
having a cavity inlet and a cavity outlet of three spiral microfluidic devices stacked in three tiers and operating in parallel with each other.

10. A method of manufacturing a spiral microfluidic module for CTC separation, the method comprising:
patterning the helical microfluidic device morphology for CTC isolation using standard UV lithography on a silicon wafer, wherein the device has two inlets with a radius of 10 mm or less, a two-loop helical microchannel having a rectangular cross-section with a uniform radial inner portion and a uniform radial outer portion, and a width of the upper portion equal to the width of the base, and the two-loop helical microchannel branching from the CTC and two outlets through which blood cells are separately discharged.
etching using reactive ion etching to form channels in the wafer;
trichloro(1H,1H,2H,2H-perfluorooctyl) silanization treatment for a predetermined time to promote mold relaxation;
curingpolydimethylsiloxane (PDMS) prepolymer after silanization;
separating the cured polydimethylsiloxane (PDMS) from the mold; and
punching holes for an inlet and an outlet in the separated polydimethylsiloxane (PDMS).

11. The method of claim 10, further comprising the step of irreversibly coupling the three polydimethylsiloxane (PDMS) devices separated in the separating step to a micro glass slide.

12. The method of claim 11, wherein in the coupling step includes laminating with plasma bonding and manual alignment.

13. The method of claim 11, wherein the coupling step includes reinforcing the coupling by placing in an oven at 65° C. to 75° C. for 25 minutes to 35 minutes.

14. The method of claim 10, further comprising washing the patterned silicon wafer using acetone and isopropanol after etching in the step of forming the channel.

15. A method for an anticancer drug reactivity test using cancer cell motility analysis to which the CTC sample isolated from the spiral microfluidic device according to any one of claims 1 to 7 is applied, the comprising:
introducing the CTC sample separated from the spiral microfluidic device for CTC separation into the chamber, wherein two inlets, each with a radius of 10 mm or less, into which blood samples and epithelial fluid requiring CTC separation are injected; a two-loop spiral microchannel having a rectangular cross section in which the radial inner portion and the radial outer portion are uniform in height and the width of the upper portion is equal to the width of the base; , including two outlets branching from the two-loop spiral microchannel to separate and discharge CTCs and blood cells; administering the anticancer agent to the chamber by setting any one or more of the type or concentration of the anticancer agent as a manipulation variable.
